(19) United States
(12) Patent Application Publication
(10) Pub. No.: US 2016/0022484 A1
(43) Pub. Date: Jan. 28, 2016

(54) OPTICAL COHERENCE TOMOGRAPHY-AUGMENTED SURGICAL INSTRUMENTS AND SYSTEMS AND METHODS FOR CORRECTING UNDESIRED MOVEMENT OF SURGICAL INSTRUMENTS

(71) Applicant: NOVARTIS AG, Basel (CH)

(72) Inventors: Lingfeng Yu, Rancho Santa Margarita, CA (US); Hugang Ren, Cypress, CA (US)

(21) Appl. No.: 14/341,752

(22) Filed: Jul. 25, 2014

(51) Int. Cl. A61F 9/007; A61B 3/10; A61B 19/00

(52) U.S. Cl. CPC A61F 9/007; A61B 19/20; A61B 3/102

(57) ABSTRACT

An OCT-augmented surgical instrument able to correct for undesired movement, as well as a system for use with such a surgical instrument and a method of correcting for undesired movement during surgery using OCT.

### BACKGROUND

1. Field of the Disclosure

The present disclosure relates to optical coherence tomography (OCT)-augmented surgical instruments and to systems and methods for correcting for undesired movement of surgical instruments using OCT.

2. Description of the Related Art

Surgery often involves precise removal of tissue or placement of incisions. In microsurgery, in particular, accurate positioning in all three dimensions as well as precise motion control is critical to avoid unintended effects, such as an inability to complete the surgery or even damage. This is especially true for ophthalmic surgeries. For example, in vitreoretinal surgeries, tool-tip positioning accuracy of around 10 μm is desired. Hand tremor is a common problem in surgeries and is difficult to avoid, yet it can regularly cause movements of a much as 50 μm. This is well outside of the desired range for vitreoretinal surgery and other microsurgeries and reduces the quality of these surgeries.

Other surgeries, such as treatment of retinal vein occlusion, are impossible to perform because movement of the surgical instruments cannot be properly controlled. Retinal vein occlusion affect 1.6% of people aged 49 and older and can be treated surgically, but currently the procedure is considered too risky to perform.

Prior approaches to combat undesired movement such as accidental movement in microsurgical procedures actively compensate for tremors. For example, one approach places a magnetometer-aided accelerometer on the surgical instrument to detect and compensate for tremors. Other approaches using proximal-end accelerometers to sense distal-end motion require complex data filtering and processing and add significant weight to the surgical instrument, which tends to cause more tremors.

Microsurgical instruments, systems, and methods that can compensate for tremors or other undesired movement in a more practical fashion are still needed.

### SUMMARY

In one embodiment, the invention relates to an OCT system containing an OCT source coupled via an OCT transmission medium to a beam splitter coupled via one OCT path to a reference arm; and via a second OCT transmission medium to an OCT-augmented surgical instrument. The OCT-augmented surgical instrument contains an OCT focusing element, an actuator, and a surgical component that performs a surgical operation. The OCT system also contains a detector coupled via an OCT transmission medium to the beam splitter. The detector receives an OCT beam containing a component from the reference arm and a component from the OCT-augmented surgical instrument. The OCT system additionally contains a computer electrically or wirelessly coupled to the detector and the actuator. An undesired movement of the surgical instrument results in a change in an interference pattern detected by the detector, which is communicated to the computer, which sends an electrical or wireless signal to the actuator to cause a corrective movement of the surgical instrument.

In another embodiment, the invention relates to an OCT-augmented surgical instrument containing an OCT transmission medium, an OCT focusing element, an actuator, and a surgical component. The actuator is able to cause corrective movement of the surgical instrument in response to an OCT beam that travels through the OCT transmission medium and OCT focusing element.

In another embodiment, the invention relates to a method of correcting undesired movement of a surgical instrument by sending an OCT beam from an OCT source via an OCT transmission medium to a beam splitter, which splits the beam into an OCT beam that travels to and is reflected by a reference arm and an OCT beam that travels to and is reflected by a tissue being operated on by the surgical instrument, detecting an interference pattern for the OCT beams reflected by the reference arm and tissue, determining whether an undesired movement occurred and an appropriate corrective movement based on the interference pattern, and

causing the appropriate corrective movement in the surgical instrument using an actuator located in the surgical instrument.

### BRIEF DESCRIPTION OF THE DRAWINGS

For a more complete understanding of the present invention and its features and advantages, reference is now made to the following description, taken in conjunction with the accompanying drawings, which are not drawn to scale, and in which:

FIG. 1 is an OCT system containing an OCT-augmented surgical instrument;

FIG. 2 is an OCT-augmented surgical instrument;

FIG. 3 is another embodiment of an OCT-augmented surgical instrument;

FIG. 4 is a beam-splitting and focusing unit in an OCT-augmented surgical instrument;

FIG. 5 is another embodiment of a beam-splitting and focusing unit in an OCT-augmented surgical instrument;

FIG. 6 is a coupler in an OCT-augmented surgical instrument; and

FIG. 7 is diagram depicting the relationship of measured vectors in space using an OCT system containing an OCT-augmented surgical instrument.

### DESCRIPTION OF PARTICULAR EMBODIMENT(S)

In the following description, details are set forth by way of example to facilitate discussion of the disclosed subject matter. It should be apparent to a person of ordinary skill in the field, however, that the disclosed embodiments are exemplary and not exhaustive of all possible embodiments.

As used herein, a reference numeral followed by a letter refers to a specific instance of an element and the numeral only form of the reference numeral refers to the collective element. Thus, for example, device ‘12a’ refers to an instance of a device class, which may be referred to collectively as devices ‘12’ and any one of which may be referred to generically as a device ‘12’.

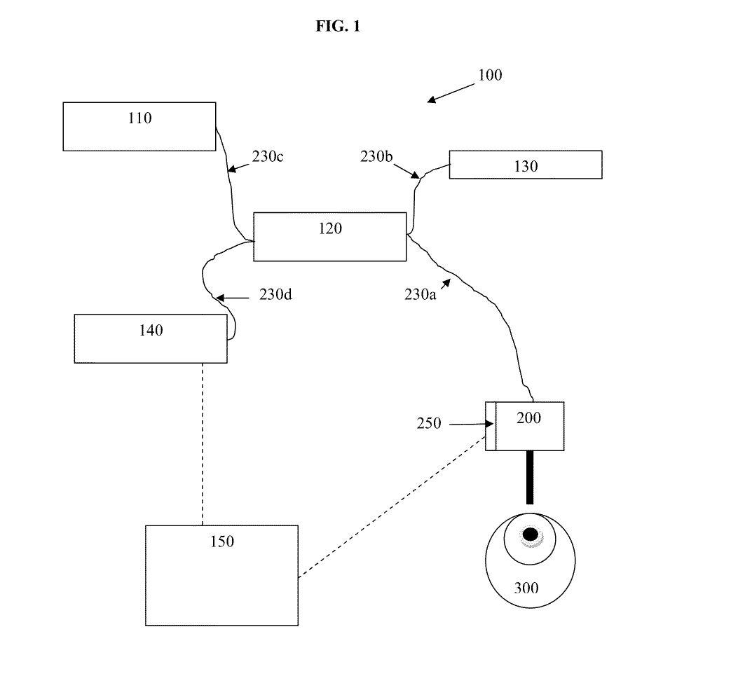

Referring now to the drawings, FIG. 1 is an OCT system 100 with OCT-augmented surgical instrument 200. Optical coherence tomography (OCT) is an interferometric analysis technique for structural examination of a sample material, such as a tissue that is at least partially reflective to light. It can also be used for functional examination of a sample material, such as the motion and velocity of the sample material or blood flow of the tissue. In OCT, light in the form on an OCT beam is used to measure distances and depth profiles based on optical interference that arises between a reference beam and a sample beam that interacts with the sample material, such as a biological tissue. In some embodiments, the OCT beam may be supplied in pulses, sweeping wavelengths or a broad band light.

OCT system 100 may make measurements of both the relative motion and velocity between surgical instrument 200 and tissue 300 (represented as an eye in this example diagram).

OCT system 100 additionally includes OCT source 110, which produces an OCT beam (not shown) that travels through OCT transmission medium 230c to beam splitter 120 where it is split so that a portion of the beam travels through OCT transmission medium 230b to reference arm 130 and a portion of the beam travels through OCT transmission medium 230a to surgical instrument 200. After hitting reference arm 130 or tissue 300, the OCT beams travel back through OCT transmission mediums 230b and 230a, respectively, to beam splitter 120, where they are directed via OCT transmission medium 230d to detector 140. Detector 140 sends a signal to computer 150, which includes a processor able to determine the relative motion and velocity of surgical instrument 200 with respect to tissue 300 based on the signal received from detector 140. Computer 150 determines if corrective movement of surgical instrument 200 is needed and, if so, sends a signal to an actuator 250 in surgical instrument 200. Actuator 250 responds to the signal and causes corrective movement of surgical instrument 200 in real-time.

In some embodiments, OCT transmission medium 230 is an optical fiber.

In the embodiment shown in FIG. 1, reference arm 130 is located close to tissue 300 in terms of optical delay and is in a pre-determined position that is an acceptable distance from the OCT source 110. The OCT beam from tissue 300 traveling back through surgical instrument 200 to detector 140, interferes with the OCT beam from reference arm 130 and generates an interference pattern. As a result, the motion characteristics (such as the gap, displacement and velocity) of the surgical instrument 200 relative to tissue 300 can be determined.

In one embodiment, reference arm 130 includes a mirror to reflect the OCT beam.

In one embodiment, detector 140 is a spectrometer. In another embodiment, detector 140 includes a photodiode or similar device that generates an electrical signal indicative of incident light intensity at detector 140.

Detector 140 may output an electrical signal to computer 150. In such an embodiment, computer 150 may include circuitry for signal conditioning, demodulation, digitization, and digital signal processing. In another embodiment, detector 140 outputs a wireless signal to computer 150.

In one embodiment, computer 150 additionally includes memory media, which store instructions (i.e., executable code) that are executable by the processor having access to the memory media. The processor may execute instructions that cause actuator 250 in surgical instrument 200 to activate and which control the parameters of such activation to allow compensation for undesired movement of surgical instrument 200. For the purposes of this disclosure, the memory media may include non-transitory computer-readable media that stores data and instructions for at least a period of time. The memory media may comprise persistent and volatile media, fixed and removable media, and magnetic and semiconductor media. The memory media may include, without limitation, storage media such as a direct access storage device (e.g., a hard disk drive or floppy disk), a sequential access storage device (e.g., a tape disk drive), compact disk (CD), random access memory (RAM), read-only memory (ROM), CD-ROM, digital versatile disc (DVD), electrically erasable programmable read-only memory (EEPROM), flash memory, non-transitory media, and various combinations of the foregoing.

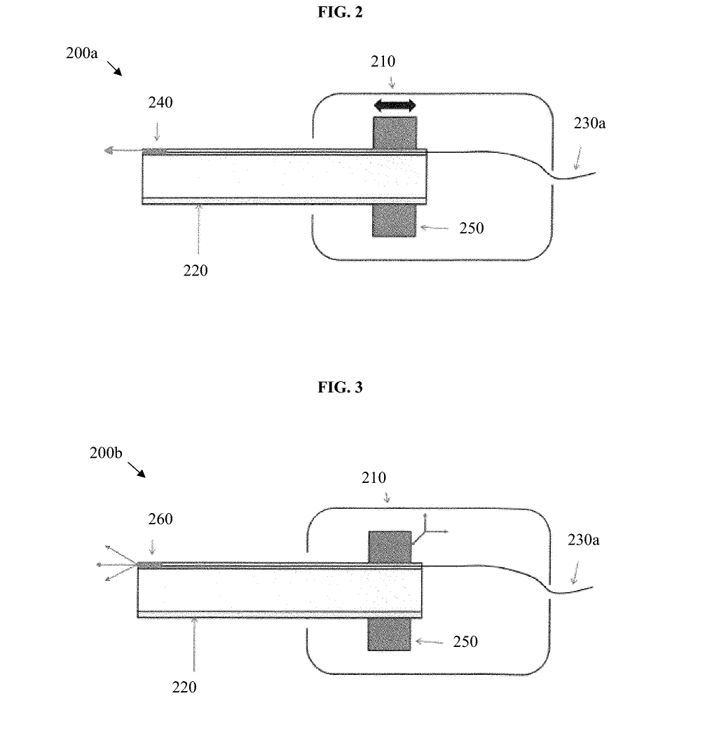

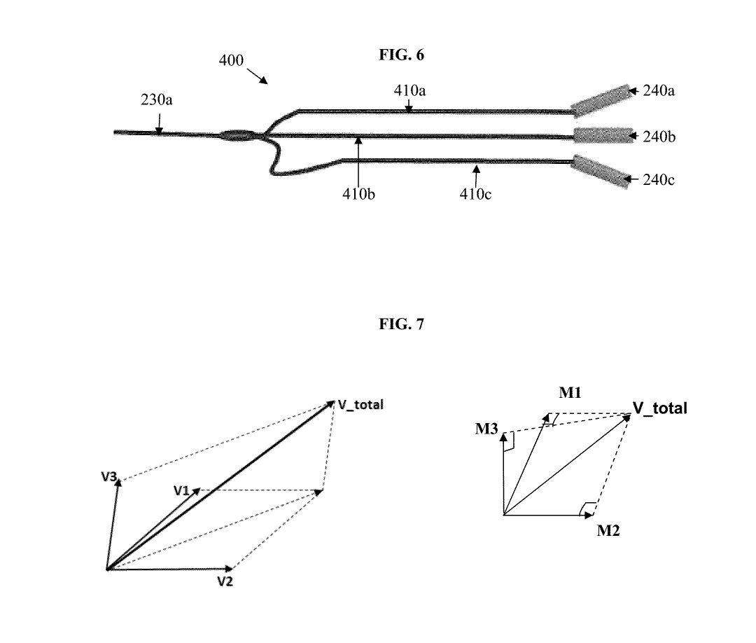

FIG. 2 depicts surgical instrument 200a, which may be used in an OCT system, and which includes handle 210 and cannula 220. In some embodiments, cannula 220 may be replaced with a different surgical component that performs a surgical operation. OCT transmission medium 230a travels through handle 210 into cannula 220, where it terminates with focusing element (e.g., lens, curved mirror) 240. Handle 210 also contains actuator 250, which is operable to receive a signal from a computer (not shown) and to cause movement of surgical instrument 200a in response to the signal. Because only one OCT beam travels through focusing element 240, the instrument in FIG. 2 provides one-dimensional OCT measurements. A surgical instrument may include multiple OCT transmission mediums and focusing lenses similar to those depicted in FIG. 2. These multiple OCT transmission mediums may be OCT fibers coupled via a coupler as shown in FIG. 6.

In the example shown, in which surgical instrument 200 contains cannula 220, actuator 250 moves cannula 220 in and out of handle 210 to compensate for undesired movement. For example, if the OCT system determines that cannula 220 is too close to the tissue (not shown), actuator 250 moves cannula 220 into handle 210 to compensate.

In some embodiments, actuator 250 moves cannula 220 or another surgical component at a speed that matches the speed of the undesired movement of surgical instrument 200. Actuator 250 may also move cannula 220 or another surgical component in a direction opposite the direction of the undesired movement, or a component direction of the undesired movement.

Actuator 250 may be any surgical actuator capable of moving surgical instrument 200 in real-time in response to undesired movement. In some embodiments, actuator 250 may constitute a small proportion of the weight of surgical instrument 200 in order to avoid introducing additional tremor. For example, actuator 250 may be less than 25% of the weight of surgical instrument 200. In one embodiment, actuator 250 is a piezoelectric actuator. In another embodiment, actuator 250 is a voice coil actuator. In still another embodiment, actuator 250 is an electromagnetic actuator. In another embodiment, actuator 250 is an ultrasonic actuator. Actuator 250 may be capable of movement in only one direction as shown in FIG. 2 or in two, three, or more directions as shown in FIG. 3, which illustrates movement in three directions. In embodiments where actuator 250 is capable of movement in two or more directions, actuator 250 may contain multiple components or sub-actuators, each capable of movement in one direction.

FIG. 3 depicts an alternative surgical instrument 200b, which, instead of focusing element 240, contains beam splitting and focusing unit 260, which splits the OCT beam traveling along OCT transmission medium 230a into two or more separate OCT beams focused in two or more directions. In one embodiment, the beam is split into three or more OCT beams focused in three or more directions. The multiple split beams produced by beam splitting and focusing unit 260 have slightly different optical path length delay, so that OCT information from different beams will be separated in depth in corresponding OCT images. Using these different images, the multi-dimensional displacement and velocity of the tissue relative to surgical instrument 200, and vice versa, is calculated.

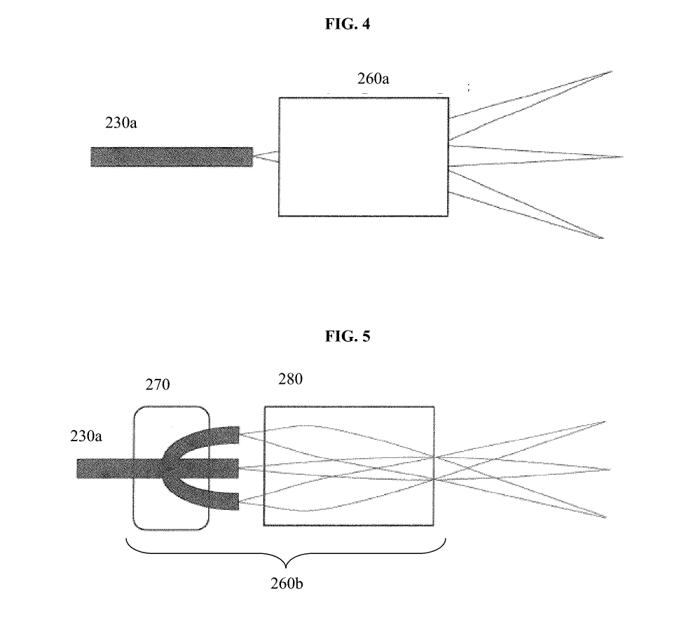

FIG. 4 depicts a unified beam splitting and focusing unit 260a. The beam splitting and focusing unit both splits the OCT beam into multiple beams and focuses those beams on the tissue (not shown).

FIG. 5 depicts an alternative beam splitting and focusing unit 260b, which contains a separate beam splitting unit 270 and beam focusing unit 280. Beam splitting unit 270, in some embodiments, is a fiber splitter or a multi-cladding fiber. Beam focusing unit 280, in some embodiments is a graded index (GRIN) lens. In other embodiments, beam splitting and focusing unit 260b is a multiple faceted ball, such as a sapphire ball.

FIG. 6 depicts a coupler 400 for splitting the OCT beam into multiple OCT fibers 410a, 410b, and 410c, which terminate in focusing elements 240a, 240b, and 240c, respectively. The multiple OCT fibers and focusing elements may be located in surgical instrument 200 in a manner similar to that depicted in FIG. 2. OCT fibers 410 may be of slightly different lengths to cause different optical path delays.

In another embodiment, the detector is able to detect polarization of light and the OCT beam through the surgical instrument is split by polarization into different orientations. This also allows multi-dimensional measurements of motion and velocity of the tissue and surgical instrument with respect to one another.

In still another embodiment, not shown, the OCT beam may be split into different spectral bands in different orientations using one or more dispersive optical elements or dichroic beam splitting optical elements. In this embodiment, the detector is able to detect the different spectral bands. This embodiment also allows multi-dimensional measurements of motion and velocity of the tissue and surgical instrument with respect to one another.

In a specific example embodiment, supplying three separate OCT beams to a tissue, FIG. 7 depicts the relationship of the beam vectors, V1, V2, and V3, in space, which represents the orientation of each beam, as well as a combined vector, V_total. The beam vectors may be displacement vectors or velocity vectors representative of a tissue with respect to a surgical instrument, and vice versa. The orientations of the beam vectors to the surgical instrument are known because they are based on instrument design or prior calibration. Note that the beam vectors, V1, V2, and V3 are the projections of the overall motion vector V_total on each beam directions. The OCT system measures the magnitude of each beam vector and calculates the overall motion vector V_total. In order to compensate the undesired motion, the surgical instrument uses multiple actuators for active motion compensation. For three-dimensional motion compensation, normally three actuators are required. Note that the orientation of the actuator motion vectors M1, M2, M3 are considered known parameters as well, but they may be not overlapping with those of the OCT beam vectors V1, V2, V3. The projection of the overall motion vector V_total onto those actuator motion directions, M1, M2, M3 can be easily calculated, and used to guide the actual motion compensation of the surgical tool.

In one embodiment, the OCT system corrects for undesired movement in the surgical instrument by sending an OCT beam from the OCT source through an OCT transmission medium to the beam splitter, which splits the OCT beam to a beam that travels to the reference arm and a beam that travels to the surgical instrument. The OCT beam in the reference arm is reflected back and travels to a detector via an OCT transmission medium, for example through the beam splitter, which may recombine it with a beam from the surgical instrument. The OCT beam in the surgical instrument is reflected back by the tissue and also travels to a detector via an OCT transmission medium, for example through the beam splitter, which may recombine it with a beam from the reference arm. The detector detects the reflected OCT beam, either as a combined beam or as components from the reference arm and surgical instrument. The detector typically detects an interference pattern, which is altered if the surgical instrument experiences a pre-determined degree of undesired movement. The detector sends an electrical or wireless signal to the computer, which then uses its processor to determine whether undesired movement has occurred and the appropriate corrective movement. The computer then sends an electrical or wireless signal to the actuator in the surgical instrument to cause corrective movement. This corrective movement may occur in real-time. For example, it may occur in less than a millisecond.

OCT-augmented surgical instruments of the types described above may be used in microsurgeries, such as vitreoretinal surgeries, including intraoccular cannulation, injection of anticoagulants to treat occlusions, and atriovenous sheathotomy, otorhinolaryngological surgeries, neurological surgeries, laproscopic surgery, prostate surgery, and microvascular surgeries. Positioning of the surgical instrument tip may be controlled to an accuracy of 10 μm or less.

The above disclosed subject matter is to be considered illustrative, and not restrictive, and the appended claims are intended to cover all such modifications, enhancements, and other embodiments which fall within the true spirit and scope of the present disclosure. Thus, to the maximum extent allowed by law, the scope of the present disclosure is to be determined by the broadest permissible interpretation of the following claims and their equivalents, and shall not be restricted or limited by the foregoing detailed description. For instance, many example embodiments herein are depicted and described using three OCT beams. It will be apparent to one of ordinary skill in the art that any plurality of OCT beams, such as three or more beams, may be used in such embodiments with corresponding increases in the complexity of calculations.

What is claimed is:

1. An optical coherence tomography (OCT) system comprising:
an OCT source coupled via a first OCT transmission medium to;
a beam splitter coupled via a second OCT transmission medium to;
a reference arm;
an OCT-augmented surgical instrument coupled via a third OCT transmission medium to the beam splitter, the surgical instrument comprising:
an OCT focusing element;
an actuator; and
a surgical component, wherein the surgical component performs a surgical operation; and
a detector coupled via a fourth OCT transmission medium to the beam splitter, wherein the detector receives an OCT beam containing a component from the reference arm and a component from the OCT-augmented surgical instrument; and
a computer electrically or wirelessly coupled to the detector and the actuator,
wherein an undesired movement of the surgical instrument results in a change in an interference pattern detected by the detector, which is communicated to the computer, which sends an electrical or wireless signal to the actuator to cause a corrective movement of the surgical instrument.

2. The OCT system of claim 1, wherein the OCT-augmented surgical instrument further comprises a splitting element that splits the OCT beam into multiple OCT beams.

3. The OCT system of claim 2, wherein the multiple OCT beams have different optical path delays.

4. The OCT system of claim 2, wherein the multiple OCT beams have different polarizations.

5. The OCT system of claim 2, wherein the multiple OCT beams have different spectral bands.

6. The OCT system of claim 2, wherein the vectors of the multiple OCT beams are used to determine the relative motion and velocity between the surgical instrument and a tissue.

7. The OCT system of claim 2, wherein the splitting element comprises a beam splitting and focusing unit that splits the OCT beam into multiple OCT beams.

8. The OCT system of claim 7, wherein the beam splitting and focusing unit comprises a separate beam splitting unit and a separate beam focusing unit.

9. The OCT system of claim 2, wherein the splitting element comprises a coupler and at least two OCT fibers.

10. The OCT system of claim 1, wherein the OCT-augmented surgical instrument contains a splitting element that splits the OCT beam into at least three OCT beams.

11. The OCT system of claim 1, wherein the focusing element comprises a focusing lens.

12. The OCT system of claim 1, wherein the focusing element comprises a curved mirror.

13. The OCT system of claim 1, wherein the surgical component comprises a cannula.

14. The OCT system of claim 1, wherein the corrective movement is in one direction.

15. The OCT system of claim 1, wherein the corrective movement is in at least two directions.

16. An optical coherence tomography (OCT)-augmented surgical instrument comprising:
an OCT transmission medium;
an OCT focusing element;
an actuator; and
a surgical component,
wherein the actuator is operable to cause corrective movement of the surgical instrument in response to an OCT beam that travels through the OCT transmission medium and OCT focusing element.

17. The instrument of claim 15, further comprising a splitting element that splits the OCT beam into multiple OCT beams.

18. The instrument of claim 16, wherein the splitting element comprises a beam splitting and focusing unit that splits the OCT beam into multiple OCT beams.

19. The instrument of claim 17, wherein the beam splitting and focusing unit comprises a separate beam splitting unit and a separate beam focusing unit.

20. The instrument of claim 16, wherein the splitting element comprises a coupler and at least two OCT fibers.

21. The instrument of claim 15, wherein the OCT focusing element comprises a focusing lens.

22. The instrument of claim 15, wherein the OCT focusing element comprises a curved mirror.

23. The instrument of claim 15, wherein the surgical component comprises a cannula.

24. A method of correcting undesired movement of a surgical instrument, the method comprising:
sending an optical coherence tomography (OCT) beam from an OCT source via a first OCT transmission medium to a beam splitter, which splits the beam into an OCT beam that travels via a second OCT transmission medium to and is reflected by a reference arm and an OCT beam that travels via a third OCT transmission medium to and is reflected by a tissue being operated on by the surgical instrument;
detecting an interference pattern for the OCT beams reflected by the reference arm and tissue;
determining whether an undesired movement occurred and an appropriate corrective movement based on the interference pattern; and
causing the appropriate corrective movement in the surgical instrument using an actuator located in the surgical instrument.

25. The method of claim 22, further comprising splitting the OCT beam that travels to the surgical instrument and determining the relative velocity and speed of the surgical instrument and the tissue.

26. The method of claim 22, wherein causing the appropriate corrective movement occurs in real-time.
